(19) United States
(12) Patent Application Publication
(10) Pub. No.: US 2017/0181821 A1
(43) Pub. Date: Jun. 29, 2017

(54) MOUTHPIECE DELIVERY APPARATUS AND METHODS OF USING THE SAME

(71) Applicant: COLGATE-PALMOLIVE COMPANY, New York, NY (US)

(72) Inventors: Scott Demarest, Basking Ridge, NJ (US); Michael Terranova, Fairfield, CT (US); Raymond Clepper, Brooklyn, NY (US)

(21) Appl. No.: 15/208,057

(22) Filed: Jul. 12, 2016

Related U.S. Application Data
(60) Provisional application No. 62/271,813, filed on Dec. 28, 2015.

(51) Int. Cl. A61C 19/06; A61C 17/02; A61M 31/00

(52) U.S. Cl. CPC A61C 19/066; A61M 31/00; A61C 17/0211

(57) ABSTRACT

A mouthpiece delivery apparatus and methods for the using the same are provided. In at least one specific embodiment, the mouthpiece delivery apparatus can include: a mouthpiece that can include a top receiving wall, a bottom receiving wall, a first side wall and a second side wall, where the bottom receiving wall can include a base member and an edge member; an adapter section, where the adapter section is configured to attach to a dispensing apparatus; and a body section, where the body section is attached to the mouthpiece at a first end and attached to the adapter section at a second end.

### CROSS-REFERENCE TO RELATED PATENT APPLICATIONS

This application claims the benefit of priority to U.S. Provisional Patent Application Ser. No. 62/271,813, filed Dec. 28, 2015, the entirety of which is incorporated herein by reference.

### BACKGROUND

There are many methods and devices for delivering oral care compositions, such as tooth whitening agents and periodontal disease treatments, to the surface of the teeth. For example, there are toothbrushes, dental trays, “strip-matrix” technologies, oral rinses, and oral sprays. However, there are many drawbacks for such methods and devices. Some require long contact times or high concentrations of ingredients, such as hydrogen peroxide, which can be injurious to the user. For oral sprays in particular, the spray may easily be misdirected to non-targeted areas such as the gums, eyes, facial tissues, and clothes.

There is a desire, therefore, to develop an improved mouthpiece delivery apparatus that can deliver oral care compositions to the surface of the teeth or other targeted areas while minimizing their exposure to non-targeted areas.

### BRIEF SUMMARY

Embodiments described herein generally relate to a mouthpiece delivery apparatus and methods of use thereof. More particularly a mouthpiece delivery apparatus that can be used to contact an oral care composition to at least a partial surface of a targeted area within the mouth, while minimizing and/or preventing contact between the oral care composition and a non-targeted area where treatment is not desired.

A mouthpiece delivery apparatus and methods of use are provided. In at least one specific embodiment, the mouthpiece delivery apparatus can include: a mouthpiece that can include a top receiving wall, a bottom receiving wall, a first side wall, and a second side wall, where the bottom receiving wall can include a base member and an edge member; an adapter section, where the adapter section is configured to attach to a dispensing apparatus; and a body section, where the body section is attached to the mouthpiece at a first end and attached to the adapter section at a second end.

In another specific embodiment, a method of using a mouthpiece delivery apparatus is disclosed that can include placing the mouthpiece delivery apparatus into a human mouth. The mouthpiece delivery apparatus can include: a mouthpiece that can include a top receiving wall, a bottom receiving wall that can include a base member and an edge member, a first side wall, and a second side wall; an adapter section configured to attach to a dispensing apparatus; and a body section that is attached to the mouthpiece at a first end and attached to the adapter section at a second end. The method can also include attaching a dispenser apparatus to the adapter section, and injecting an oral care composition from the dispenser apparatus into the mouthpiece delivery apparatus to at least partially coat a surface of an oral member.

### BRIEF DESCRIPTION OF DRAWINGS

So that the manner in which the above recited features of the present invention can be understood in detail, a more particular description of the invention, briefly summarized above, can be had by reference to embodiments, some of which are illustrated in the appended drawings. It is to be noted, however, that the appended drawings illustrate only typical embodiments of this invention and are therefore not to be considered limiting of its scope, for the invention can admit to other equally effective embodiments.

FIG. 1 shows a perspective view of a specific embodiment of the mouthpiece delivery apparatus.

FIG. 2 shows a bisected side view of a specific embodiment of the mouthpiece delivery apparatus.

FIG. 3 shows a perspective view of another specific embodiment of the mouthpiece delivery apparatus.

FIG. 4 shows a perspective view of another specific embodiment of the mouthpiece delivery apparatus.

### DETAILED DESCRIPTION

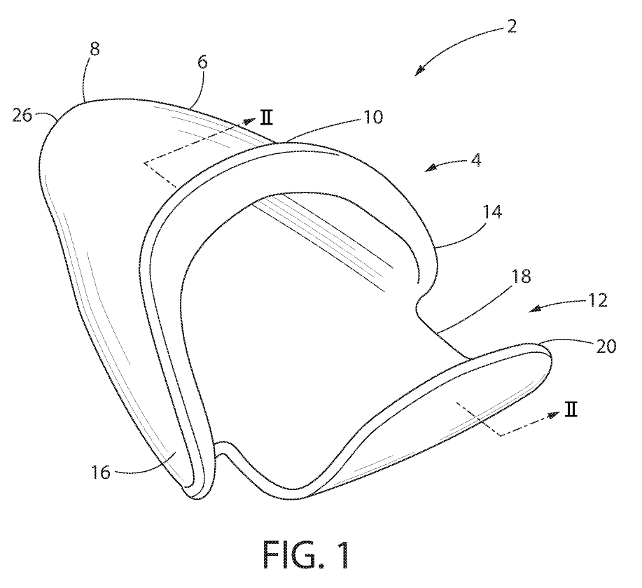

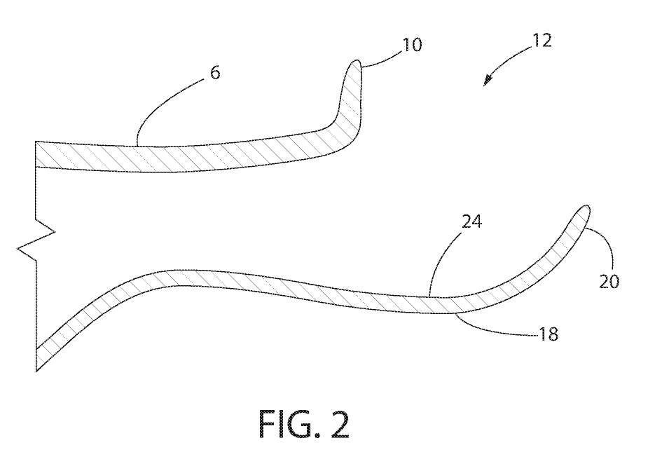

Specific embodiments of the mouthpiece delivery apparatus 2 are shown in FIGS. 1-4. The mouthpiece delivery apparatus 2 can include a mouthpiece 4, a body section 6, and an adapter section 8. The mouthpiece 4 can be adjacent to and integrally connected with a first end of the body section 6. A second end of the body section 6 can be adjacent to and integrally connected with the adapter section 8. The mouthpiece 4, the body section 6, and the adapter section 8 can include a tubular shape and be in fluid communication with each other, allowing for the directing and/or transferring of an oral care composition, such as a liquid, powder, and/or gas, to least a partial surface of a targeted area within the mouth, while minimizing and/or preventing contact between the oral care composition and a non-targeted area where treatment is not desired. The targeted area within the mouth can include, but is not limited to: an oral member; such as one or more teeth, or the gingiva (gums), such as the marginal gingiva, the attached gingiva, and/or the interdental gingiva; a surface of a tooth; or any combination thereof. The non-targeted area of the mouth can include, but is not limited: the soft tissues; the lips; the gingiva (gums), such as the marginal gingiva, the attached gingiva, and/or the interdental gingiva; the cheek mucosa, the soft palate, the hard palate, the floor of the mouth, tongue, the tongue, and any combination thereof.

When placed into the mouth of a human user, the mouthpiece delivery apparatus 2 can guide a quantity of an oral care composition to the user's teeth. The oral care composition can be delivered via a variety of ways, such as adhesive forces and/or capillary action, along the inner walls, or a mechanism which propels the oral care composition through the mouthpiece. In some embodiments, the mouthpiece 4 and the body section 6 can rotate around a central axis of the adapter section 8 at a joint where the body section 6 attaches to the adapter section 8 so both the upper teeth and the lower teeth can be treated oral care composition.

### Mouthpiece

The mouthpiece 4 can be adapted to comfortably fit the dental arch of the user and accommodate a variety of dental arches. Dental arches can vary from person to person; however, a general mouthpiece configuration can be used fit a majority or even a large percentage of the population. The mouthpiece 4 can include a top receiving wall 10, a bottom receiving wall 12, a first side wall 14, and a second side wall 16. The top receiving wall 10, the bottom receiving wall 12, the first side wall 14, and the second side wall 16 can be integrally connected to form a tubular or hollow shape, which can also be integrally connected and lie adjacent to the body section 6 of the mouthpiece delivery apparatus 2. The top receiving wall 10 and the bottom receiving wall 12 are adapted to generally conform and receive the upper teeth and/or the lower teeth of the mouth. The top receiving wall 10 can have a U-shape to fit the circumference of the upper gingivia and/or buccal or labial face of the upper teeth. The top receiving wall 10 can also have a U-shape to fit around the circumference of the lower gingivia and/or buccal or labial face of the lower teeth. The top receiving wall 10 can also have a U-shape to fit around the circumference of both the upper and the lower gingivia and/or buccal or labial face of the teeth for embodiments of the mouthpiece delivery apparatus 4 that can rotate for use on both the upper and the lower teeth. The top receiving wall 10 can guide the mouthpiece delivery apparatus 2 into the mouth of the user by resting on the gingiva, the marginal gingiva, and/or the teeth themselves, such as the buccal and/or labial faces. The top receiving wall 10 can conform to the gums and/or the teeth, masking and/or blocking non-targeted areas of the mouth to minimize and/or prevent the oral care composition from contacting these surfaces. In other words, the top receiving wall 10 can be placed or positioned in a user's mouth on the gums, the teeth, or the intersection of the gums and teeth, allowing the mouthpiece delivery apparatus to direct and/or transfer an oral care composition to the surface of the teeth, thereby at least partially coating a surface of a tooth, which may include hard to reach spaces such as in between adjacent teeth, while blocking and/or minimizing contact or touching between the oral care composition and non-targeted areas, such as the lips, the gums, or the tongue.

The first side wall 14 and the second side wall 16 can be contoured to allow the user to wrap their lips around the mouthpiece 4. The first side wall 14 and the second side wall 16 can have mirror symmetry with one another or can be asymmetric. The first side wall 14 and the second side wall 16 can block and/or minimize contact of the oral care composition with non-targeted areas, such soft tissues including the lips, the cheeks, and the gums.

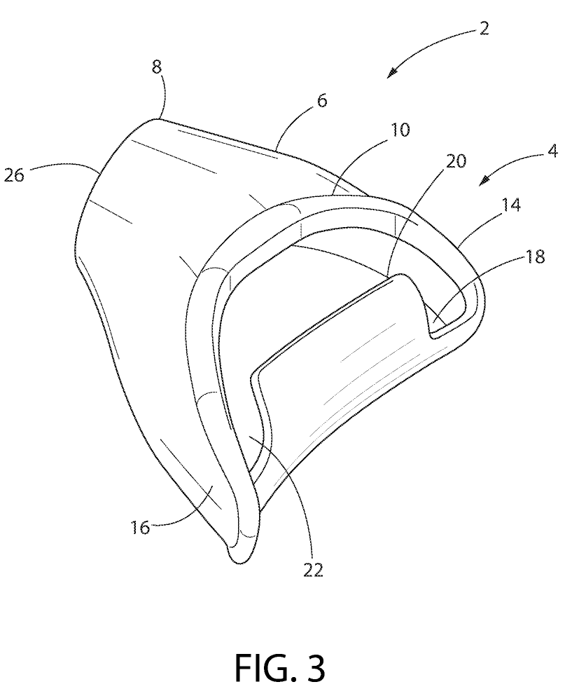

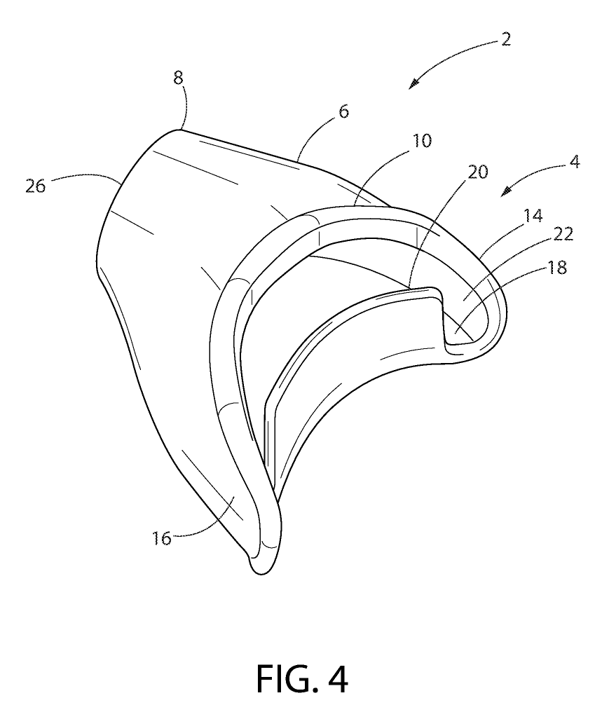

The bottom receiving wall 12 can include a base member 18 and an edge member 20. The base member 18 and the edge member 20 can be adjacent and integrally connected. The contour of the edge member 20 can include recesses 22 that can allow space for the side teeth to fit when in use. The base member 18 of the mouthpiece can pass just under the occlusal or ridge of the upper teeth when the mouthpiece delivery apparatus 2 is used to for the upper teeth. For embodiments that fit the bottom teeth or can be rotated to fit the bottom teeth, the base member 18 can pass just over the occlusal or ridge of the bottom teeth. In some embodiments, the side teeth do not contact the edge member 20 at the recesses 22. For example, the mouthpiece delivery apparatus 2 can be held in place by contacting the user at the top receiving wall 10, the first side wall 14, and the second side wall 16. In other embodiments, the side teeth can at least partially contact the edge member 20 at the recesses 22 (see, e.g., FIGS. 3 and 4). For example, a user can position the mouthpiece delivery apparatus 2 in their mouth and hold it in place by contacting mouth surfaces at the top receiving wall 10, the first side wall 14, the second side wall 16, and the edge member 20 at the recesses 22. In some embodiments, the top receiving wall 10 can also act as a guide that helps informing the user as to how to orient the mouthpiece delivery apparatus 2 inside his or her mouth.

In certain embodiments, the edge member 20 can have a U-shape to generally follow the circumference of the lingual or palatal faces of the teeth (see, e.g., FIG. 4). In other embodiments, the edge member 20 can have a straight configuration (see, e.g., FIG. 3). In certain embodiments, such as those depicted in FIGS. 1-4, the edge member 20 can curve upward from the base member 18 toward the roof of the mouth when used for the upper teeth. This upward curve of the edge member 20 can minimize and/or prevent the oral care composition from contacting the tongue and/or other soft tissue surfaces where treatment is not desired. For example, if the oral care composition is sprayed onto the user's upper teeth, the edge member 20 can curve up behind the user's teeth to block and/or catch any excess spray from entering the mouth and contacting the soft tissues, such as the tongue. The base member 18 can extend any length that would fit in the user's mouth. The edge member 20 can extend upward to any length that would fit in the user's mouth. The edge member 20 can curve upward from the base member 18 at any angle from 1° to 179°.

In certain embodiments, the base member 18 and the edge member 20 can have lengths and an angle that would position the edge member 20, when in use, to lie just behind a lingual and/or palatal face of one or more teeth (see, e.g., FIG. 3). In other embodiments, the base member 18 and the edge member 20 can have lengths and an angle that would place the edge member 20, when in use, in contact with a lingual and/or palatal face of one or more teeth (see, e.g., FIG. 4). In certain embodiments, the base member 18 and the edge member 20 can have lengths and an angle that would place the edge member 20, when in use, to lie adjacent to a hard palate or a surface of a hard palate. In other embodiments, the base member 18 and the edge member 20 can have lengths and an angle that would place the edge member 20, when in use, in contact with a hard palate or a surface of a hard palate. In certain embodiments, the base member 18 and the edge member 20 can have lengths and an angle that would place the edge member 20, when in use, to lie adjacent to a soft palate or a surface of a soft palate. In other words, the base member 18 and the edge member 20 can have lengths and an angle that point the edge member 20 towards the roof of the mouth. In other embodiments, the base member 18 and the edge member 20 can have lengths and an angle that would place the edge member 20, when in use, in contact with a soft palate or a surface of a soft palate.

In certain embodiments, the edge member 20 and/or the base member 18 can block and/or catch at least a portion of the oral care composition. For example, the oral care composition can be injected and/or propelled into the mouthpiece delivery apparatus 2 to at least partial contact and/or coat an oral member, while the excess oral care composition is block and/or caught by the edge member 20 and/or the base member 18.

The mouthpiece can be configured to receive any desired number of teeth. Using the Universal Numbering System the mouthpiece can be configured to envelop any of teeth 1 through 32. For example, the mouthpiece can be configured to envelop teeth 5 through 12. In some embodiments, the same configuration can then be rotated to envelop teeth 21 through 28.

In certain embodiments, the mouthpiece delivery apparatus 2 can be placed or positioned in the mouth or configured to allow the oral care composition to be direct and/or transferred to targeted specific areas of the gingiva. For example, the top receiving wall 10 can be placed or positioned on the gingiva to allow for at least a partial contacting of the marginal gingiva and/or the interdental gingiva with oral care composition. The mouthpiece delivery apparatus 2 can be placed or positioned in the mouth or configured to allow for the oral care composition to be direct and/or transferred to treat oral diseases, such gingivitis and periodontitis.

### Body Section

The body section 6 can include a wall with an inner face and an outer face forming a tubular shape to aid in directing and/or transferring an oral care composition from the adapter section 8 to the mouthpiece 4 and finally onto the surface of the user's teeth. The tubular shape of the body section 6 can have various shapes. For example, the tubular shape of the body section 6 when cross-sectioned parallel to the mouthpiece 4 and the adapter section 8 can form a symmetric closed curve or an asymmetric closed curve. For another example, the tubular shape of the body section 6 when cross-sectioned parallel to the mouthpiece and the adapter section can form a circle, oval, ellipse, vesica piscis, rectangle, or any other desired geometric shape, etc. In specific embodiments, the body section 6 can have one or more sides with a flat profile. In other specific embodiments, the body section 6 can have one or more sides with a convex profile, forming a cavity 24 to allow for the pooling of any excess oral care composition that drips from the teeth or the edge member 20. The body section 6 can have an increasing, decreasing, both increasing and decreasing, or constant volume along the direction of travel of the oral care composition.

### Adapter Section

The adapter section 8 can allow for the attachment of a dispensing apparatus (not shown) that can inject an oral care composition into the mouthpiece delivery apparatus 2. The dispensing apparatus can be, but is not limited to: aerosol spray canisters, tubes, pumps, bottles, piezeo spray devices, and ultrasonic systems. The adapter section 8 can be configured to attach to the dispensing apparatus to allow for the oral care composition to be transferred (e.g., sprayed) through the mouthpiece delivery apparatus 2 to at least partially coat the surface of a user's tooth. The dispensing apparatus can be attached to the adapter section 8 at any angle with respect to the mouthpiece delivery apparatus 2. In some embodiments, the adapter section 8 can have a joint where the body section 6 attaches to the adapter section 8 that allows for the mouthpiece 4 and the body section 6 to rotate around a central axis of the adapter section 8.

In specific embodiments, the adapter section 8 can have a cone shape. In other specific embodiments, the adapter section 8 can have one or more cylindrical walls. For embodiments with a plurality of cylindrical walls, the cylindrical walls can lie adjacent to and be integrally connected with each other in a series centered on their rotational axes. The plurality of cylindrical walls can have an increasing radii, decreasing radii, or sections along the series with both increasing and decreasing radii. In other embodiments, the adapter section 8 can assume any other desired geometric shapes.

The adapter section 8 can include a terminal wall 26 at the end that attaches to the dispensing apparatus. The terminal wall 26 can have one or more ports to allow for the passage of the oral care composition from dispensing apparatus into the mouthpiece delivery apparatus 2. In specific embodiments, the one or more ports can direct or modulate the oral care composition as it enters from the dispensing apparatus.

### Materials

The mouthpiece delivery apparatus 2 can be made of any suitable material. For example, the mouthpiece delivery apparatus 2 can be made out of one or more plastics, one or more thermoplastic elastomers, one or more polymers, one or more metals, one or more alloys, one or more silicones, one or more rubbers, and any mixture or combination thereof. The mouthpiece delivery apparatus 2 can be made from the same material or more than one different materials. For example, the mouthpiece 4 can be made from a first material, the body section 6 can be made from a second material, and the adapter section 8 can be from a third material. The individual structural elements of the mouthpiece delivery apparatus 2 can also be made from the same material or more than one different materials. For example, the top receiving wall 10 and the bottom receiving wall 12 of the mouthpiece can be made from a first material and the first side wall 14 and the second side wall 16 of the mouthpiece can be made from a second material.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility. It is therefore intended by the appended claims to cover any and all such applications, modifications and embodiments within the scope of the present invention.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

What is claimed is:

1. A mouthpiece delivery apparatus comprising:
a mouthpiece comprising a top receiving wall, a bottom receiving wall, a first side wall and a second side wall, wherein the bottom receiving wall comprises a base member and an edge member;
an adapter section, wherein the adapter section is configured to attach to a dispensing apparatus; and
a body section, wherein the body section is attached to the mouthpiece at a first end and attached to the adapter section at a second end, and wherein the mouthpiece, the adapter section, and the body section.

2. The mouthpiece delivery apparatus as in claim 1, wherein the edge member is positioned behind a lingual surface of one or more teeth when the mouthpiece delivery apparatus is in a human mouth.

3. The mouthpiece delivery apparatus as in claim 1, wherein the edge member is positioned adjacent to a soft palate when the mouthpiece delivery apparatus is in a human mouth.

4. The mouthpiece delivery apparatus as in claim 1, wherein the bottom receiving wall comprises recesses that do not touch side teeth when the mouthpiece delivery apparatus is in a human mouth.

5. The mouthpiece delivery apparatus as in claim 1, wherein the bottom receiving wall comprises recesses that touch side teeth when the mouthpiece delivery apparatus is in a human mouth.

6. The mouthpiece delivery apparatus as in claim 1, wherein the body section comprises a tubular shape.

7. The mouthpiece delivery apparatus as in claim 1, wherein the body section and the mouthpiece rotate around a central axis of the adapter section at a joint where the body section attaches to the adapter section.

8. The mouthpiece delivery apparatus as in claim 1, wherein the extension member catches at least a portion of an oral care composition.

9. A method of using a mouthpiece delivery apparatus comprising:
placing the mouthpiece delivery apparatus into a human mouth,
wherein the mouthpiece delivery apparatus comprises:
a mouthpiece comprising:
a top receiving wall;
a bottom receiving wall comprising a base member and an edge member;
a first side wall; and
a second side wall;
an adapter section configured to attach to a dispensing apparatus; and
a body section that is attached to the mouthpiece at a first end and attached to the adapter section at a second end;
wherein the mouthpiece, the adapter section, and the body section;
attaching a dispenser apparatus to the adapter section; and
injecting an oral care composition from the dispenser apparatus into the mouthpiece delivery apparatus to at least partially coat a surface of an oral member.

10. The method as in claim 9, wherein the oral member comprises a tooth, a gingiva, or any combination thereof.

11. The method as in claim 9, wherein the edge member is positioned behind a lingual surface of a tooth when the mouthpiece delivery apparatus is in a human mouth.

12. The method as in claim 9, wherein the edge member is positioned adjacent to a soft palate when the mouthpiece delivery apparatus is in a human mouth.

13. The method as in claim 9, wherein the edge member is positioned to touch a surface of a soft palate when the mouthpiece delivery apparatus is in a human mouth.

14. The method as in claim 9, wherein the body section comprises a cavity.

15. The method as in claim 9, wherein the bottom receiving wall comprises recesses that touch side teeth when the mouthpiece delivery apparatus is in a human mouth.
